(19) United States
(12) Patent Application Publication
(10) Pub. No.: US 2012/0226160 A1
(43) Pub. Date: Sep. 6, 2012

(54) ULTRASOUND DIAGNOSTIC APPARATUS AND ULTRASOUND IMAGE PRODUCING METHOD

(75) Inventor: Yoshimitsu KUDOH, Kanagawa (JP)

(73) Assignee: FUJIFILM CORPORATION, Tokyo (JP)

(21) Appl. No.: 13/347,296

(22) Filed: Jan. 10, 2012

(30) Foreign Application Priority Data
Mar. 3, 2011 (JP) 2011-046061

(51) Int. Cl. A61B 8/13

(52) U.S. Cl. 600/443

(57) ABSTRACT

An ultrasound diagnostic apparatus comprises: an ultrasound probe which performs transmission and reception of ultrasonic beams using a transducer array according to a mode selected by an operator from a low image quality mode and a high image quality mode, and which processes reception signals outputted from the transducer array in reception signal processors to generate digital reception data; a diagnostic apparatus body for producing an ultrasound image based on the reception data transmitted from the ultrasound probe and displaying the produced ultrasound image on a monitor; a temperature detecting unit for detecting an internal temperature of the ultrasound probe, and an uptime manager for calculating an uptime in the high image quality mode based on the internal temperature of the ultrasound probe detected by the temperature detecting unit to display the calculated uptime on the monitor.

### BACKGROUND OF THE INVENTION

The present invention relates to an ultrasound diagnostic apparatus and an ultrasound image producing method. The invention more particularly relates to an ultrasound diagnostic apparatus for making a diagnosis based on ultrasound images produced by transmitting and receiving ultrasonic waves from and in a transducer array of an ultrasound probe.

Conventionally, ultrasound diagnostic apparatus using ultrasound images are employed in the medical field. In general, this type of ultrasound diagnostic apparatus comprises an ultrasound probe having a built-in transducer array and an apparatus body connected to the ultrasound probe. The ultrasound probe transmits ultrasonic waves toward a subject and receives ultrasonic echoes from the subject, and the apparatus body electrically processes the reception signals to generate an ultrasound image.

In such ultrasound diagnostic apparatus, the transducer array transmits ultrasonic waves to generate heat.

An operator usually make a diagnosis as he or she holds the ultrasound probe in a single hand and places the ultrasound transmission/reception surface of the transducer array in contact with a subject's skin and therefore the ultrasound probe is often encased in a housing of such a small size that the operator can readily hold it in a single hand. Therefore, the heat generated in the transducer array may raise the temperature inside the housing of the ultrasound probe.

In recent years, there has been proposed an ultrasound diagnostic apparatus having an ultrasound probe with a built-in circuit board for signal processing and performing digital processing of reception signals outputted from the transducer array before transmitting the reception signals to the apparatus body via wireless or wired communication thereby reducing the effects of noise and obtaining a high-quality ultrasound image.

In the ultrasound probe that performs this type of digital processing, heat is generated from the circuit board also during the processing of the reception signals, and therefore the temperature rise in the housing needs to be suppressed to assure stable operations of the circuits on the board.

As one of the measures against the increase in the ultrasound probe temperature, for example, JP 2005-253776 A discloses an ultrasound diagnostic apparatus in which conditions for driving the transducer array are automatically changed in accordance with the surface temperature of the ultrasound probe to switch the mode of ultrasound images to be acquired from high image quality to low image quality, thereby keeping the surface of the ultrasound probe at an appropriate temperature. More specifically, the surface temperature of the ultrasound probe is kept at an appropriate temperature by reducing, for example, the drive voltage of each transducer of the transducer array for the transmission of ultrasonic waves, the number of simultaneously available channels for transmission, the transmission pulse repetition frequency and the frame rate with increasing surface temperature of the ultrasound probe.

However, when the surface temperature of the ultrasound probe is increased by heat released from the transducer array and the circuit board during the operation in the mode for acquiring high quality ultrasound images, that is, the high image quality mode, the apparatus of JP 2005-253776 A may automatically switch to the mode for acquiring low quality ultrasound images, that is, the low image quality mode despite the operator's intentions.

### SUMMARY OF THE INVENTION

The present invention has been made to solve the foregoing prior art problems and an object of the invention is to provide an ultrasound diagnostic apparatus capable of easily knowing how long more the operator can continue the diagnostic operation in the high image quality mode. Another object of the invention is to provide an ultrasound image producing method used in the ultrasound diagnostic apparatus.

An ultrasound diagnostic apparatus according to the present invention comprises:

an ultrasound probe which performs transmission and reception of ultrasonic beams using a transducer array according to a mode selected by an operator from a low image quality mode and a high image quality mode, and which processes reception signals outputted from the transducer array in reception signal processors to generate digital reception data;

a diagnostic apparatus body for producing an ultrasound image based on the reception data transmitted from the ultrasound probe and displaying the produced ultrasound image on a monitor;

a temperature detecting unit for detecting an internal temperature of the ultrasound probe, and

an uptime manager for calculating an uptime in the high image quality mode based on the internal temperature of the ultrasound probe detected by the temperature detecting unit to display the calculated uptime on the monitor.

An ultrasound image producing method according to the present invention comprises the steps of:

performing transmission and reception of ultrasonic beams using a transducer array of an ultrasound probe according to a mode selected by an operator from a low image quality mode and a high image quality mode;

processing reception signals outputted from the transducer array in reception signal processors to generate digital reception data;

producing an ultrasound image in a diagnostic apparatus body based on the reception data transmitted from the ultrasound probe and displaying the produced ultrasound image on a monitor;

detecting an internal temperature of the ultrasound probe; and

calculating an uptime in the high image quality mode based on the detected internal temperature of the ultrasound probe to display the calculated uptime on the monitor.

### BRIEF DESCRIPTION OF THE DRAWINGS

FIG. 1 is a block diagram showing the configuration of an ultrasound diagnostic apparatus according to Embodiment 1.

FIG. 2 is a diagram showing a screen on which the uptime in the high image quality mode according to Embodiment 1 is displayed.

FIG. 3 is a diagram showing the uptime in the high image quality mode.

FIG. 4 is a diagram showing the screen when the uptime in the high image quality mode decreases to a preset value or less.

FIG. 5 is a block diagram showing the configuration of an ultrasound probe that may be used in the ultrasound diagnostic apparatus according to Embodiment 2.

### DETAILED DESCRIPTION OF THE INVENTION

Embodiments of the present invention will be described below based on the accompanying drawings.

### Embodiment 1

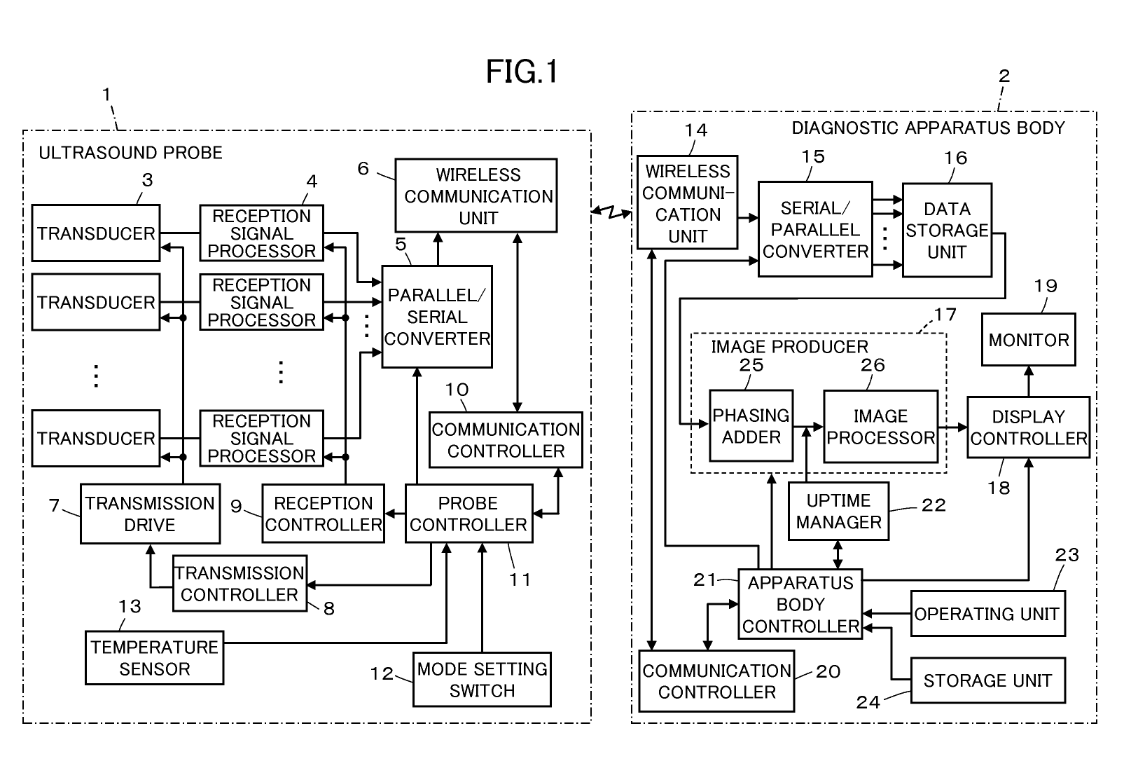

FIG. 1 shows the configuration of an ultrasound diagnostic apparatus according to Embodiment 1 of the invention. The ultrasound diagnostic apparatus comprises an ultrasound probe 1 and a diagnostic apparatus body 2 that is connected to the ultrasound probe 1 via wireless communication.

The ultrasound probe 1 comprises a plurality of ultrasound transducers 3 constituting a plurality of channels of a one-dimensional or two-dimensional transducer array, and the transducers 3 are connected to their corresponding reception signal processors 4, which in turn are connected to a wireless communication unit 6 via a parallel/serial converter 5. The transducers 3 are connected to a transmission controller 8 via a transmission drive 7, the reception signal processors 4 are connected to a reception controller 9, and the wireless communication unit 6 is connected to a communication controller 10. The parallel/serial converter 5, the transmission controller 8, the reception controller 9, and the communication controller 10 are connected to a probe controller 11. Operators can select one of the low image quality mode and high image quality mode, and a mode setting switch 12 provided on a housing of the ultrasound probe 1 is connected to the probe controller 11. The ultrasound probe 1 also has a built-in temperature sensor 13 for detecting the internal temperature T of the ultrasound probe 1, and the temperature sensor 13 is connected to the probe controller 11.

The temperature sensor 13 is preferably disposed near the reception signal processors 4 where heat is expected to develop during the operation of the ultrasound diagnostic apparatus.

The transducers 3 each transmit ultrasonic waves according to drive signals supplied from the transmission drive 7 and receive ultrasonic echoes from the subject to output reception signals. Each of the transducers 3 includes a vibrator having a piezoelectric body made of, for example, a piezoelectric ceramic material typified by PZT (lead zirconate titanate), a piezoelectric polymer typified by PVDF (polyvinylidene fluoride) or a piezoelectric single crystal typified by PMN-PT (lead magnesium niobate-lead titanate solid solution), and electrodes provided at both ends of the piezoelectric body.

When the electrodes of the vibrator are supplied with a pulsed voltage or a continuous-wave voltage, the piezoelectric body expands and contracts to cause the vibrator to produce pulsed or continuous ultrasonic waves. These ultrasonic waves are combined to form an ultrasonic beam. Upon reception of propagating ultrasonic waves, each vibrator expands and contracts to produce electric signals, which are then outputted as ultrasonic reception signals.

The transmission drive 7 includes, for example, a plurality of pulsers and adjusts the delay amounts of drive signals for the respective transducers 3 based on a transmission delay pattern selected by the transmission controller 8 so that the ultrasonic waves transmitted from the transducers 3 form an ultrasonic beam, thereby supplying the transducers 3 with adjusted drive signals.

Under the control of the reception controller 9, the reception signal processor 4 in each channel subjects the reception signals outputted from the corresponding transducer 3 to quadrature detection or quadrature sampling to produce complex baseband signals, samples the complex baseband signals to generate sample data containing information on the area of the tissue, and supplies the parallel/serial converter 5 with the sample data. The reception signal processors 4 may generate the sample data by performing data compression for highly efficient coding on the data obtained by sampling the complex baseband signals.

The parallel/serial converter 5 converts the parallel sample data generated by the reception signal processors 4 in a plurality of channels into serial sample data.

The wireless communication unit 6 performs carrier modulation based on the serial sample data to generate transmission signals and supplies an antenna with the transmission signals so that the antenna transmits radio waves to transmit the serial sample data. The modulation methods that may be employed herein include ASK (Amplitude Shift Keying), PSK (Phase Shift Keying), QPSK (Quadrature Phase Shift Keying), and 16QAM (16 Quadrature Amplitude Modulation).

The wireless communication unit 6 transmits the sample data to the diagnostic apparatus body 2 through wireless communication with the diagnostic apparatus body 2, receives various control signals from the diagnostic apparatus body 2, and outputs the received control signals to the communication controller 10. The communication controller 10 controls the wireless communication unit 6 so that the sample data is transmitted at a transmission radio field intensity that is set by the probe controller 11 and outputs various control signals received by the wireless communication unit 6 to the probe controller 11.

The temperature sensor 13 detects the internal temperature T of the ultrasound probe 1 and outputs it to the probe controller 11.

The probe controller 11 controls various components of the ultrasound probe 1 according to various control signals transmitted from the diagnostic apparatus body 2. The probe controller 11 also controls the number of simultaneously available channels of the transducer array for the reception according to the mode selected with the mode setting switch 12.

The ultrasound probe 1 has a built-in battery (not shown) which supplies electric power to the circuits inside the ultrasound probe 1.

The ultrasound probe 1 may be of an external type such as linear scan type, convex scan type or sector scan type, or of, for example, a radial scan type used in an ultrasound endoscope.

On the other hand, the diagnostic apparatus body 2 comprises a wireless communication unit 14, which is connected to a data storage unit 16 via a serial/parallel converter 15. The data storage unit 16 is connected to an image producer 17. The image producer 17 is connected to a monitor 19 via a display controller 18. The wireless communication unit 14 is also connected to a communication controller 20 and the serial/parallel converter 15, the image producer 17, the display controller 18, and the communication controller 20 are connected to an apparatus body controller 21. The apparatus body controller 21 is connected to an uptime manager 22 for calculating the time period for which the operation in the high image quality mode is continued (this time period is hereinafter referred to as “uptime”) based on the internal temperature T of the ultrasound probe 1. The apparatus body controller 21 is connected to an operating unit 23 for an operator to perform input operations and to a storage unit 24 for storing operation programs.

The wireless communication unit 14 transmits various control signals to the ultrasound probe 1 through wireless communication with the ultrasound probe 1. The wireless communication unit 14 demodulates the signals received by the antenna to output serial sample data.

The communication controller 20 controls the wireless communication unit 14 so that various control signals are transmitted at a transmission radio field intensity that is set by the apparatus body controller 21.

The serial/parallel converter 15 converts the serial sample data outputted from the wireless communication unit 14 into parallel sample data. The data storage unit 16 is constituted by a memory, a hard disk, or the like and stores at least one frame of sample data converted by the serial/parallel converter 15.

The image producer 17 performs reception focusing on each frame of sample data read out from the data storage unit 16 to generate image signals representing an ultrasound diagnostic image. The image producer 17 includes a phasing adder 25 and an image processor 26.

The phasing adder 25 selects one reception delay pattern from a plurality of previously stored reception delay patterns according to the reception direction set by the apparatus body controller 21 and, based on the selected reception delay pattern, provides the complex baseband signals represented by the sample data with respective delays and adds them up to perform the reception focusing. This reception focusing yields baseband signals (sound ray signals) where the ultrasonic echoes are well focused.

The image processor 26 generates B-mode image signals, which are tomographic image information on a tissue inside the subject, according to the sound ray signals generated by the phasing adder 25. The image processor 26 includes an STC (sensitivity time control) section and a DSC (digital scan converter). The STC section corrects the sound ray signals for the attenuation due to distance according to the depth of the reflection position of the ultrasonic waves. The DSC converts the sound ray signals corrected by the STC into image signals compatible with the scanning method of ordinary television signals (raster conversion), and generates B-mode image signals through required image processing such as gradation processing. The image processor 26 also produces image signals and character signals on the uptime in the high image quality mode which was calculated by the uptime manager 22.

The display controller 18 causes the monitor 19 to display an ultrasound diagnostic image according to the image signals generated by the image producer 17. The monitor 19 includes a display device such as an LCD, for example, and displays an ultrasound diagnostic image under the control of the display controller 18.

The uptime manager 22 calculates the uptime in the high image quality mode based on the internal temperature T of the ultrasound probe 1 detected by the temperature sensor 13.

The apparatus body controller 21 controls the components in the diagnostic apparatus body 2.

While the serial/parallel converter 15, the image producer 17, the display controller 18, the communication controller 20, and the apparatus body controller 21 in the diagnostic apparatus body 2 are each constituted by a CPU and an operation program for causing the CPU to perform various kinds of processing, they may be constituted by a digital circuit. The operation program is stored in the storage unit 24. Exemplary recording media that may be used in the storage unit 24 in addition to the built-in hard disk include a flexible disk, an MO, an MT, an RAM, a CD-ROM and a DVD-ROM.

The number of channels for the reception in the low image quality mode is set so that, of the total number of channels of the transducer array, a predetermined number of channels are simultaneously available.

On the other hand, the number of channels for the reception in the high image quality mode is set so that, of the total number of channels of the transducer array, a larger number of channels than the predetermined number of channels set as the low image quality mode are simultaneously available.

When the transducer array has, for example, 48 channels in total, the number N of simultaneously available channels for the reception is set to 24 or 32 channels when the low image quality mode is selected and 48 channels when the high image quality mode is selected.

The number of simultaneously available channels for the reception in each mode may be previously entered from the operating unit 23 of the diagnostic apparatus body 2 and be stored in the storage unit 24 as a table of the number of simultaneously available channels.

As for the transmission, ultrasonic waves are transmitted using all the channels of the transducer array irrespective of the selected mode.

Next, the operation of Embodiment 1 will be described.

Prior to the diagnosis, an operator uses the mode setting switch 12 to select high image quality mode or low image quality mode. The selected mode is wirelessly transmitted to the diagnostic apparatus body 2 via the probe controller 11, the communication controller 10 and the wireless communication unit 6.

The apparatus body controller 21 reads out a table of the number of simultaneously available channels stored in the storage unit 24 and set the number of simultaneously available channels for the reception based on the selected mode. The number of simultaneously available channels is wirelessly transmitted from the apparatus body controller 21 to the ultrasound probe 1 via the communication controller 20 and the wireless communication unit 14 and inputted to the probe controller 11 via the wireless communication unit 6 and the communication controller 10 of the ultrasound probe 1.

The probe controller 11 operates the transmission drive 7 via the transmission controller 8, and ultrasonic waves are transmitted from the transducers 3 in all the channels of the transducer array according to the drive signals supplied from the transmission drive 7. Thus, the reception signals are outputted from the transducers 3 having received ultrasonic echoes from the subject. In this process, the probe controller 11 controls the reception signal processors 4 via the reception controller 9 so that the channels, the number of which has been set according to the mode selected by the operator, may be simultaneously available.

The reception signals from the transducers 3 for which the number of simultaneously available channels is set in each mode are supplied to the corresponding reception signal processors 4 to generate sample data, which is converted into serial data in the parallel/serial converter 5 before being transmitted wirelessly from the wireless communication unit 6 to the diagnostic apparatus body 2. The sample data received by the wireless communication unit 14 of the diagnostic apparatus body 2 is converted into parallel data in the serial/parallel converter 15 and stored in the data storage unit 16. Further, the sample data is read out from the data storage unit 16 frame by frame to generate image signals in the image producer 17. The display controller 18 causes the monitor 19 to display an ultrasound image based on the image signals.

When the ultrasound diagnostic apparatus is thus operated, the internal temperature T of the ultrasound probe 1 is detected by the built-in temperature sensor 13 of the ultrasound probe 1. The internal temperature T is wirelessly transmitted to the diagnostic apparatus body 2 via the probe controller 11, the communication controller 10 and the wireless communication unit 6. The internal temperature T received by the wireless communication unit 14 of the diagnostic apparatus body 2 is inputted to the apparatus body controller 21 via the communication controller 20 and is further transmitted from the apparatus body controller 21 to the uptime manager 22.

The uptime manager 22 calculates the uptime in the high image quality mode based on the transmitted internal temperature T of the ultrasound probe 1. As shown in formula 1 below, the uptime in the high image quality mode is calculated from, for example, the internal temperature T detected by the temperature sensor 13, the power consumption value of the ultrasound probe 1 calculated from the amount of the current supplied to each component of the ultrasound probe 1 in the high image quality mode, and the heat capacity determined from the structure of the ultrasound probe 1.

Uptime in the high image quality mode=k3*[k2−probe internal temperature T−k1(probe power consumption value/probe heat capacity)](k1, k2 and k3 are constants.) (Formula 1)

The thus calculated uptime in the high image quality mode is sent to the image producer 17, and image signals and character signals are generated in the image processor 26. The display controller 18 causes the monitor 19 to display an ultrasound diagnostic image.

It is thus possible to detect the internal temperature T of the ultrasound probe 1 with the temperature sensor 13, calculate the uptime in the high image quality mode in the uptime manager 22 based on the internal temperature T and display the calculated uptime on the monitor 19. Therefore, the operator can appropriately and easily confirm the uptime in the high image quality mode as he or she makes a diagnosis. The operator can also arrange the subsequent imaging schedule according to the remaining uptime to make the diagnosis without causing unintentional stopping of the imaging in the high image quality mode and mode switching.

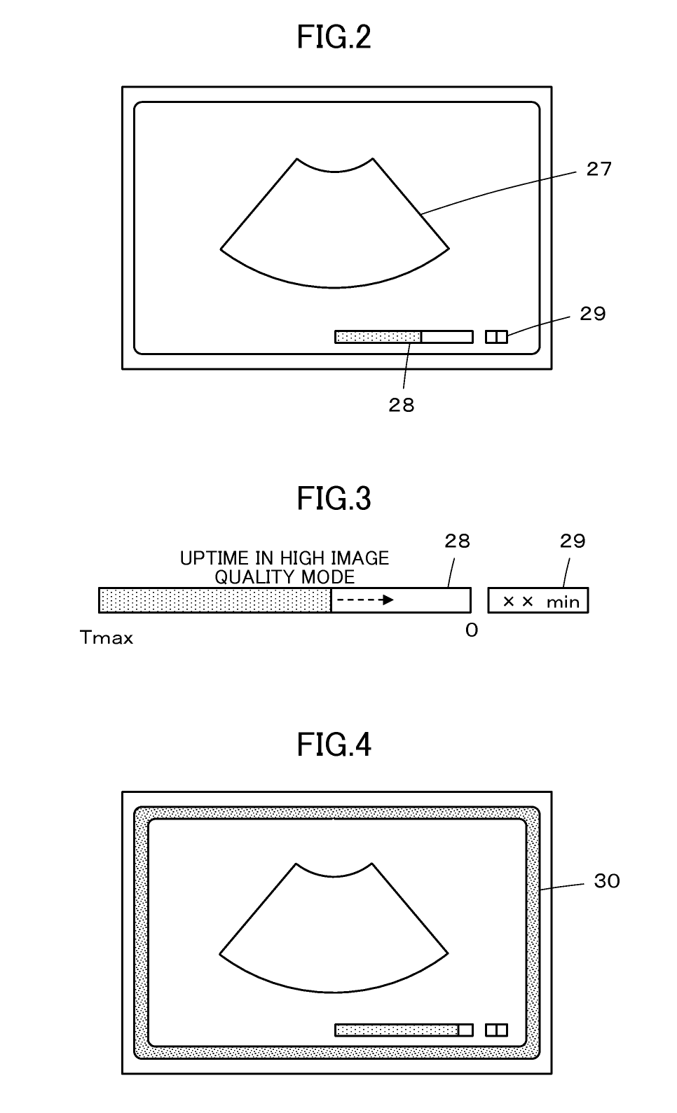

An exemplary screen on which the uptime in the high image quality mode is displayed together with an ultrasound image is shown in FIG. 2. The uptime is displayed below an ultrasound image 27 using a color bar 28 and a numerical value 29.

The color bar 28 represents the uptime in the high image quality mode as a ratio to a given time period. As shown in FIG. 3, as the uptime decreases, the colored portion increases in the direction indicated by an arrow in FIG. 3, that is, from “Tₘₐₓ” showing the maximum uptime toward the direction of “0” indicating that the operation cannot be continued any more. By displaying the uptime in the high image quality mode with the color bar 28 as described above, the operator who is at a position more or less distant from the display screen can also easily and visually know the time period for which imaging can be made in the high image quality mode if he or she can see the display screen from this position. The color bar 28 may be marked with a scale so that the remaining uptime can be more clearly known.

The numerical value 29 shows the remaining uptime. The display of the numerical value enables the operator to clearly know the remaining uptime for which the operation in the high image quality mode can be performed.

In Embodiment 1, the uptime in the high image quality mode is displayed on the screen irrespective of whether the high image quality mode or the low image quality mode is selected. However, this is not the sole case of the invention and the uptime may be displayed only when the high image quality mode is selected. During the diagnosis in the high image quality mode, the operator can continue the diagnosis while checking how long more he or she can capture high-quality ultrasound images and therefore it is possible to set the subsequent diagnostic schedule, for example, as to whether to continue the imaging in the high image quality mode or whether to change the image quality mode from the high image quality mode to the low image quality mode.

Even in cases where the low image quality mode is selected, the uptime in the high image quality mode is preferably displayed on the screen when it decreases to a preset value or less. This is because the operator can continue the diagnosis in the high image quality mode for a time period the operator desires after switching from the low image quality mode to the high image quality mode.

When the uptime in the high image quality mode decreases to a preset value or less in Embodiment 1, the periphery 30 of the screen can be displayed in color as shown in FIG. 4. Such a display is effective to inform the operator that the uptime in the high image quality mode is running out, whereby the operator can correspondingly take prompt measures such as changing the mode.

The foregoing screen display which invites the operator's attention is not limited to this. The periphery 30 of the screen may be displayed in color in a flashing manner or the color of the periphery 30 of the screen may be changed stepwise in accordance with the remaining uptime.

In Embodiment 1, the number of simultaneously available channels for the reception is controlled to switch the image quality mode between the high image quality mode and the low image quality mode but this is not the sole case of the invention. The mode may be switched by controlling the frame rate, the number of sound rays per frame and measured depth.

### Embodiment 2

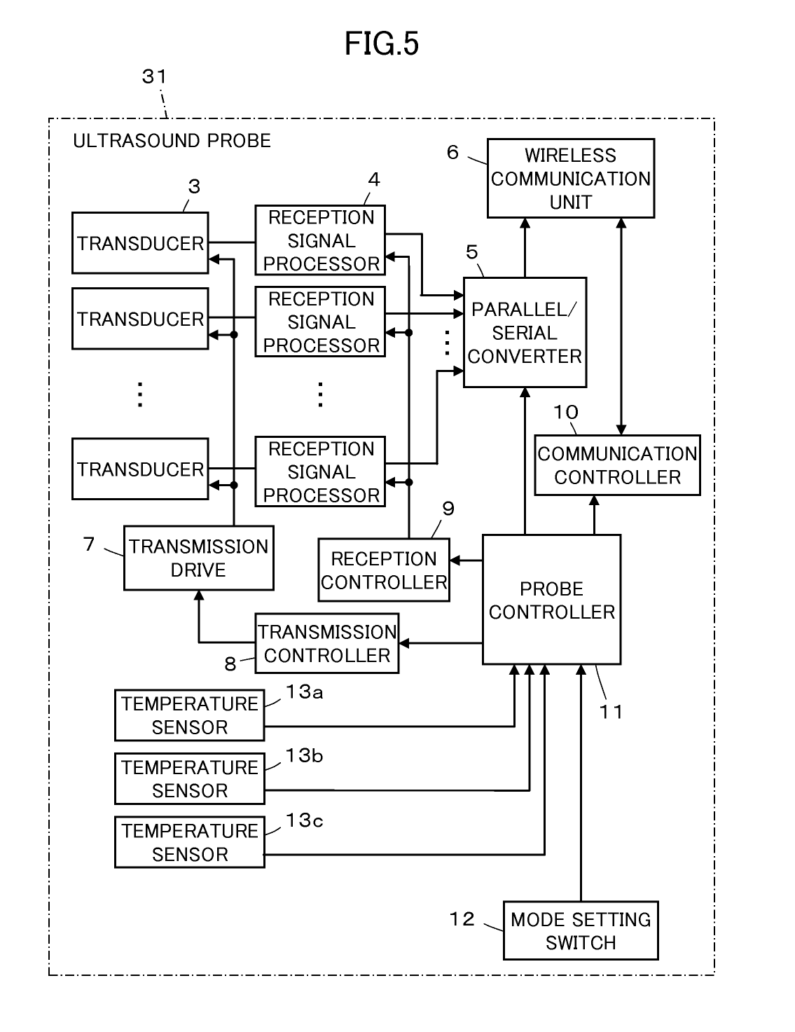

FIG. 5 shows the configuration of an ultrasound probe 31 that may be used in the ultrasound diagnostic apparatus according to Embodiment 2. The ultrasound probe 31 is obtained by providing the ultrasound probe 1 of Embodiment 1 shown in FIG. 1 with a plurality of temperature sensors 13a to 13c instead of the temperature sensor 13.

The temperature sensors 13a to 13c are connected to the probe controller 11. These temperature sensors 13a to 13c are preferably disposed, for example, near the reception signal processors 4, near the transducer array (not shown), on the periphery of the battery, at the housing of the ultrasound probe 31 held by the operator or near a member where heat is expected to develop during the operation.

The temperature values detected by the temperature sensors 13a to 13c are wirelessly transmitted from the probe controller 11 to the diagnostic apparatus body 2 via the communication controller 10 and the wireless communication unit 6. The temperature values received by the wireless communication unit 14 of the diagnostic apparatus body 2 are inputted to the apparatus body controller 21 via the communication controller 20 and are further transmitted from the apparatus body controller 21 to the uptime manager 22.

The temperature detected by the temperature sensors inside the ultrasound probe 31 varies with where the temperature sensors are disposed. The maximum temperature (maximum withstand temperature) up to which members disposed inside the ultrasound probe 31 can stably operate depends on the members. Of the values detected by the temperature sensors 13a to 13c and received by the uptime manager 22, the uptime manager 22 uses, as the internal temperature T of the ultrasound probe 31, one of the values which is the closest to the maximum withstand temperature preset in the places where the temperature sensors are disposed, and calculates the uptime in the high image quality mode.

The maximum withstand temperature is preferably set to be less than 40° C. when the temperature sensor is disposed near the transducer array, less than 38° C. when it is disposed at the housing held by the operator, and less than 60° C. when it is disposed near the reception signal processors 4.

The temperature near the members where heat is expected to develop during the operation can be detected by providing the temperature sensors 13a to 13c in plural places inside the ultrasound probe 31. The uptime is calculated based on the temperatures at which the members inside the ultrasound probe 31 can stably operate so that the operator can be informed of more precise uptime in the high image quality mode.

What is claimed is:

1. An ultrasound diagnostic apparatus comprising:
an ultrasound probe which performs transmission and reception of ultrasonic beams using a transducer array according to a mode selected by an operator from a low image quality mode and a high image quality mode, and which processes reception signals outputted from the transducer array in reception signal processors to generate digital reception data;
a diagnostic apparatus body for producing an ultrasound image based on the reception data transmitted from the ultrasound probe and displaying the produced ultrasound image on a monitor;
a temperature detecting unit for detecting an internal temperature of the ultrasound probe, and
an uptime manager for calculating an uptime in the high image quality mode based on the internal temperature of the ultrasound probe detected by the temperature detecting unit to display the calculated uptime on the monitor.

2. The ultrasound diagnostic apparatus according to claim 1, further comprising:
a mode setting switch disposed in the ultrasound probe and used to select one of the low image quality mode and the high image quality mode; and
a controller for controlling the reception signal processors so that a number of simultaneously available channels is limited to a first predetermined value when the low image quality mode is selected by the mode setting switch and to a second predetermined value which is larger than the first predetermined value when the high image quality mode is selected by the mode setting switch.

3. The ultrasound diagnostic apparatus according to claim 1, wherein the uptime manager causes the monitor to display the calculated uptime only when the high image quality mode is selected.

4. The ultrasound diagnostic apparatus according to claim 1, wherein the uptime manager causes the monitor to display the calculated uptime in a form of a color bar.

5. The ultrasound diagnostic apparatus according to claim 1, wherein the uptime manager changes a color of a periphery of a screen in the monitor when the calculated uptime decreases to a preset value or less.

6. The ultrasound diagnostic apparatus according to claim 1, wherein the temperature detecting unit comprises a temperature sensor.

7. The ultrasound diagnostic apparatus according to claim 1,
wherein the temperature detecting unit comprises temperature sensors for detecting the internal temperature at different positions of the ultrasound probe, and
wherein the uptime manager calculates the uptime based on values of the internal temperature detected by the temperature sensors.

8. An ultrasound image producing method comprising the steps of:
performing transmission and reception of ultrasonic beams using a transducer array of an ultrasound probe according to a mode selected by an operator from a low image quality mode and a high image quality mode;
processing reception signals outputted from the transducer array in reception signal processors to generate digital reception data;
producing an ultrasound image in a diagnostic apparatus body based on the reception data transmitted from the ultrasound probe and displaying the produced ultrasound image on a monitor;
detecting an internal temperature of the ultrasound probe; and
calculating an uptime in the high image quality mode based on the detected internal temperature of the ultrasound probe to display the calculated uptime on the monitor.
